(19) United States
(12) Patent Application Publication
(10) Pub. No.: US 2024/0090932 A1
(43) Pub. Date: Mar. 21, 2024

(54) Guidewire Retention Mechanism

(71) Applicants: Kevin T. Foley, Germantown, TN (US); Jeff Justis, Germantown, TN (US)

(72) Inventors: Kevin T. Foley, Germantown, TN (US); Jeff Justis, Germantown, TN (US)

(21) Appl. No.: 18/060,269

(22) Filed: Nov. 30, 2022

Related U.S. Application Data
(60) Provisional application No. 63/284,920, filed on Dec. 1, 2021.

(51) Int. Cl. A61B 17/88; A61B 17/17

(52) U.S. Cl. CPC A61B 17/8897; A61B 17/1757

(57) ABSTRACT

A cannulated surgical instrument having a cannula lock configured to selectively lock and unlock a guidewire extending into the instrument's cannula. The instrument includes a cannulated shaft having a cannulated driving member attached to a proximal end of the cannulated shaft. The cannula lock is configured to selectively move between (i) a locked position where the guidewire is engaged and cannot move axially with respect to the cannula, and (ii) an unlocked position where the guidewire is disengaged and can move axially with respect to the cannula.

### CROSS REFERENCE TO RELATED APPLICATION

This application claims the benefit under 35 USC § 119(e) of U.S. Provisional Application Ser. No. 63/284,920 filed Dec. 1, 2021, which is incorporated by reference herein in its entirety.

### BACKGROUND

Many surgical procedures are assisted through employment of a guidewire used in conjunction with cannulated instruments. Guidewires are commonly used in various spinal procedures such as the placement of hardware (e.g., pedicle screws) in the vertebrae. Precise control of guidewire placement and particularly depth penetration is critical to avoid neural damage and injury to major blood vessels proximate to the vertebrae. These spinal procedures often require penetration of the hard cortical bone of the vertebra and traversal of the softer cancellous bone lying thereunder. A large force is normally required by the surgeon to penetrate the cortical bone. Once the cortical bone is penetrated, extreme care must then be taken to avoid rapidly penetrating through all of the cancellous bone, and possibly even the cortical bone on the other side of the vertebrae.

There are many instances during the procedure where the cannulated instrument must be free to slide relative to the guidewire position through the instrument's cannulation. However, there are also times where the surgeon is required to simultaneously control manipulation of the cannulated instrument and control the relative position (depth) of the guidewire because it is not fixed relative to the instrument. This arrangement increases the risk that the guidewire may move beyond the instrument's leading tip and penetrate to a depth not anticipated or intended by the surgeon while the surgeon is focused on manipulation of the instrument. It would be a significant improvement in the art to provide a surgical instrument which can selectively lock and unlock the guidewire's current position in the cannula, thus ensuring the guidewire cannot be extended further beyond the instrument's leading tip than intended. This improvement avoids certain problems encountered in prior art methods of employing guidewires. For example, when a tap (or other cannulated instrument) is inserted over a guidewire (e.g., K-wire) that has been placed into the bone prior to tapping the bone, the tap (or other cannulated instrument) can be inadvertently directed along a trajectory that is slightly “off axis” relative to the K-wire, resulting in binding of the K-wire within the cannulated instrument being inserted over it. This binding of the K-wire can result in the K-wire being inadvertently driven deeper, even to the point that the K-wire is driven through the bone into an adjacent anatomic space (for example, into the retroperitoneal space and peritoneal cavity). By locking the K-wire to the tap (or other cannulated instrument), the invention precludes this undesirable outcome. Likewise, if a conventional cannulated tap is advanced beyond the distal end of the K-wire, cancellous bone can enter the distal portion of the cannula and bind the K-wire inside the cannula. When the tap is then withdrawn, the bone within the cannula tip can “push” the K-wire and inadvertently remove it. Being able to lock the K-wire such that its end remains flush with the distal end of the cannula (or protrudes slightly beyond the distal end of the cannula) prevents bone from inadvertently entering the distal end of the cannula and precludes this phenomenon.

### SUMMARY

One embodiment of the invention is a cannulated surgical instrument having a cannula lock configured to selectively lock and unlock a guidewire extending into the instrument's cannula. The instrument includes a cannulated shaft having a cannulated driving member attached to a proximal end of the cannulated shaft. The cannula lock is configured to selectively move between (i) a locked position where the guidewire is engaged and cannot move axially with respect to the cannula, and (ii) an unlocked position where the guidewire is disengaged and can move axially with respect to the cannula. The cannula lock may take on many different designs, including those described in the following disclosure.

### BRIEF DESCRIPTION OF THE DRAWINGS

FIG. 1 is a perspective view of one embodiment of the surgical instrument used in conjunction with an instrument guide which is attached to the arms of a surgical robot.

FIG. 2 is a perspective view of an adapter insert for the surgical guide having a smaller diameter (for smaller diameter instruments) than the surgical guide itself.

FIG. 3 is a perspective view of the surgical instrument (absent the handle) positioned above the instrument guide with a first marker array connected to the instrument and a second marker array connected to the instrument guide.

FIG. 4 is a cross-sectional view of the instrument handle.

FIG. 5A is a more detailed cross-sectional view of the instrument handle illustrated in FIG. 4.

FIG. 5B is a cross-sectional view of a first alternative cannula lock.

FIGS. 5C and 5D are views of a second alternative cannula lock.

FIGS. 5E to 5G are views of a third alternative cannula lock.

FIGS. 5H and 5I are views of a fourth alternative cannula lock.

FIG. 6 is a perspective view of the surgical instrument being inserted through the instrument guide with the cannula lock in the locked position to hold the guidewire in place.

FIG. 7 is a cross-sectional view of a vertebra showing the trajectory of an instrument into the vertebral body.

FIG. 8 is a perspective view of the guide wire being left in engagement with the vertebra after the cannula lock has been placed in the unlocked position and the surgical instrument pulled off the guidewire.

FIG. 9 is a perspective view of a cannulated drill bit.

FIG. 10 is a side view of a conventional cannulated power drill.

### DETAILED DESCRIPTION

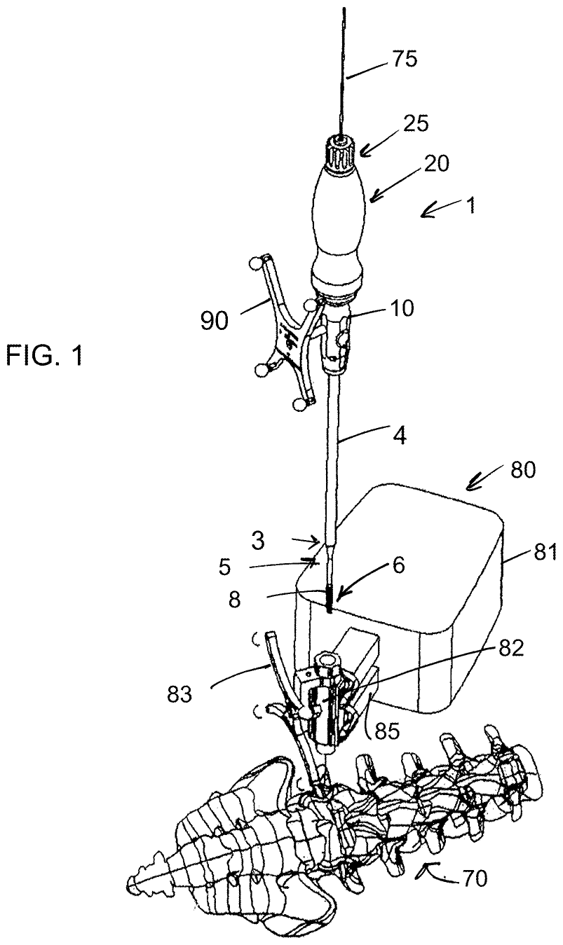

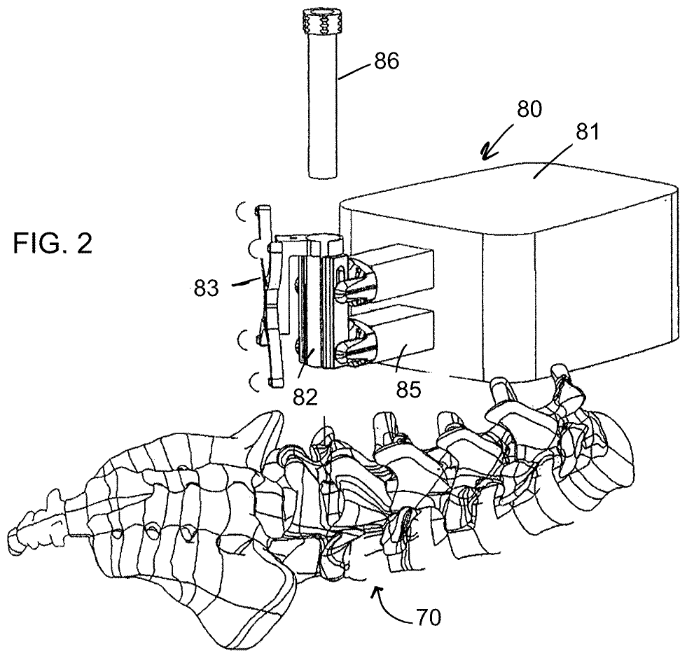

FIG. 1 illustrates one example of the cannulated surgical instrument 1 of the present disclosure, together with a navigation system/surgical robot 80 with which the surgical instrument 1 could be used. The spinal column 70 is one example of the anatomy on which a procedure using surgical instrument 1 could be performed. However, neither the surgical instrument 1 nor methods described herein are limited to spinal procedures. It will also be understood that navigation/robot system 80 does not form part of the present invention, but is a conventional surgical system suggesting one manner in which surgical instrument 1 could be employed. For the purposes of this disclosure, navigation/robot system 80 generally comprises targeting platform 81, instrument guide 82 with its tracking array 83, and robotic arm 85. FIG. 2 more clearly shows an adapter insert 86 commonly used with instrument guide 82. In the illustrated embodiment, navigation/robot system 80 could be a Micromate™ personal robotic assistant system available from iSYS Medizintechnik GmbH, of Kitzbühel, Austria (d/b/a Interventional Systems). The tracking array 83 allows a camera(s) of a conventional surgical navigation system to determine the position and orientation of the surgical instrument. One such camera could be a monocular localization camera available from Intellijoint Surgical, Inc. of Kitchener, Canada and the navigation technique could be that described in international application no. PCT/US2020/056013 filed Oct. 16, 2020, which is incorporated by reference herein in its entirety.

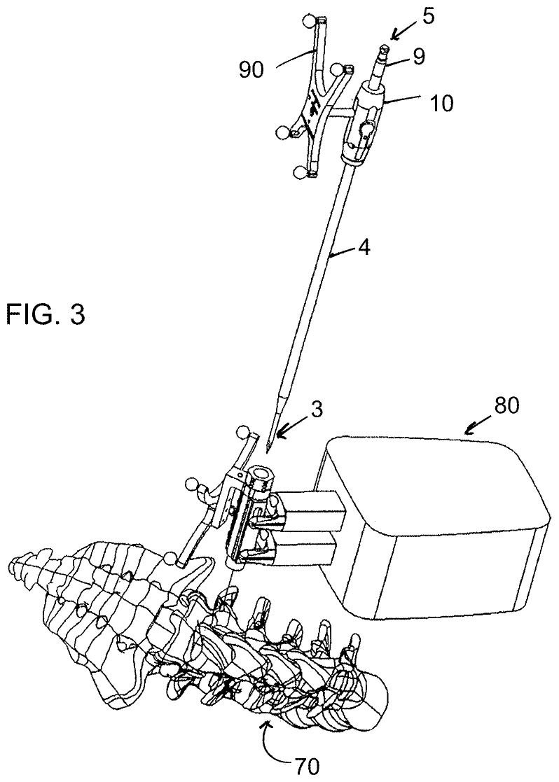

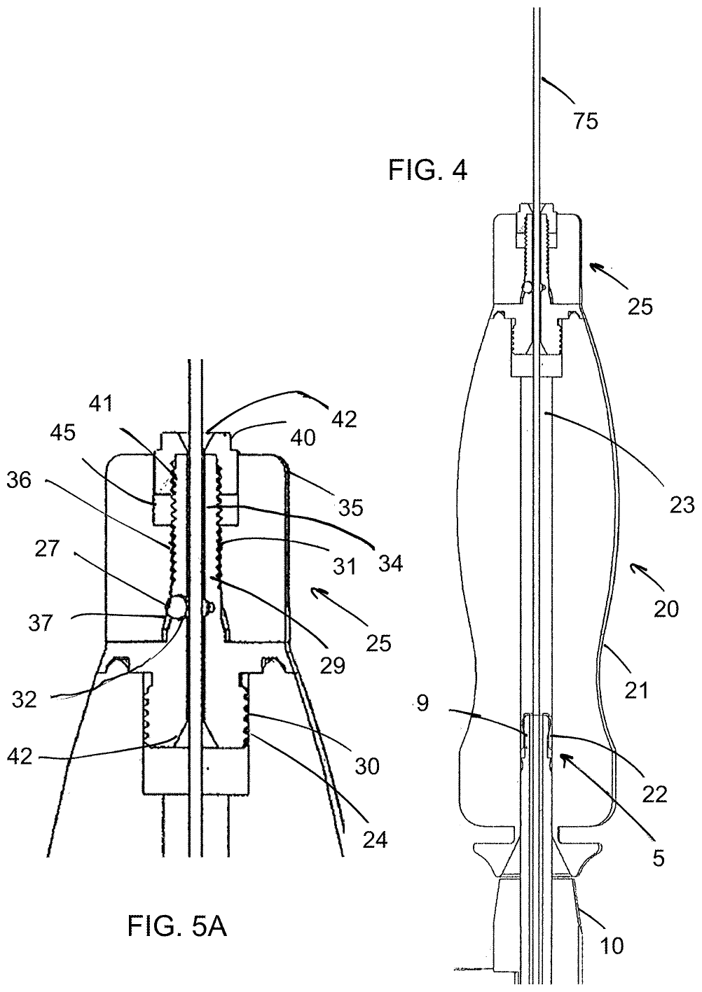

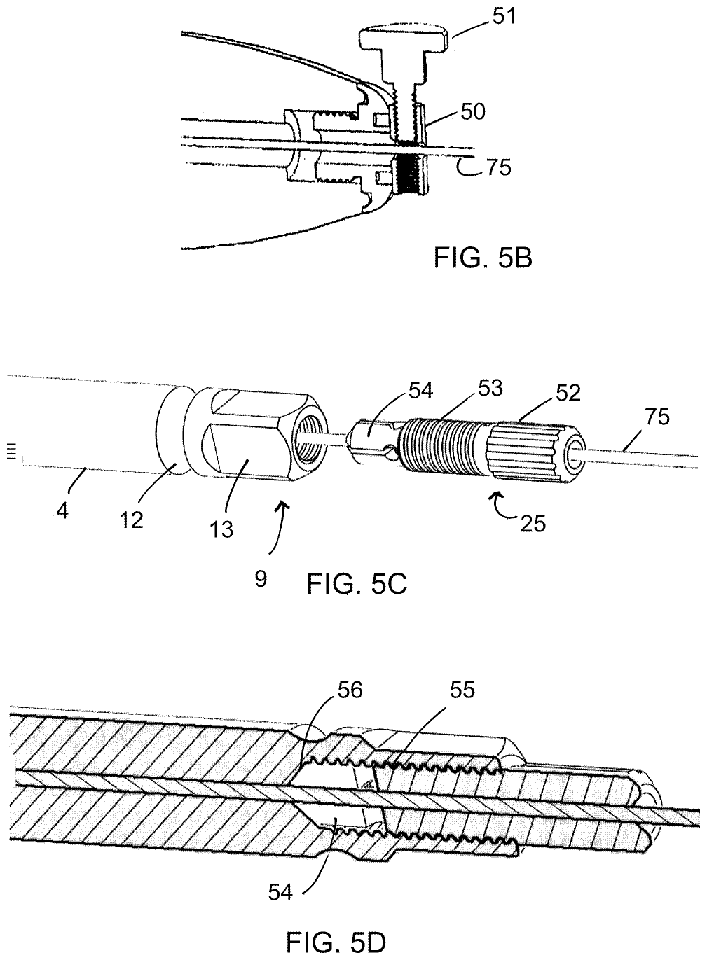

The cannulated surgical instrument 1 generally comprises the cannulated shaft 4 connected to cannulated handle 20 with the mounting collar 10 positioned below handle 20. This example of mounting collar 10 includes the tracking array 90 which enables the navigational camera to track the position and orientation of the surgical instrument. The instrument central passage is sized to allow free passage of conventional guidewire sizes, such as guidewires varying from 0.014 to 0.038 inches in diameter in one non-limiting example. As seen in more detail in FIG. 4, an upper or proximal end 5 of shaft 4 will include the shaft's handle connector 9 which engages the handle's shaft connector 22. In the illustrated embodiment, the shaft's handle connector 9 is formed by the square drive-head 13 and locking ring 12 (best seen in FIGS. 3 and 5C) which engages the handle's shaft connector 22. Although not shown in the figures, the handle's shaft connector 22 could include a socket for receiving square drive-head 13 with spring loaded balls configured to engage the locking ring 12 on the shaft's handle connector 9. A movable collar on the handle's shaft connector 22 would (i) in a first position holds the balls in locking ring 12, and (ii) in a second position allow the balls to retract out of locking ring 12. The socket of shaft connector 22 engages square drive-head 13 such that the shaft 4 is fixed to the handle 20 in a manner that there is no relative rotation of the shaft and handle. Above handle connector 22 is the handle grip section 21 which extends to the cannula lock 25 seen at the top (proximal end) of handle 20 in this embodiment. As best seen in FIG. 5A, cannula lock 25 (also sometimes referred to as a “locking mechanism”) generally includes the locking knob 35 engaging the lock mandrel 29. The mandrel central passage 34 extends through lock mandrel 29. Lock mandrel 29 connects to the top of handle 20 via the external lower mandrel threads 30 engaging corresponding internal handle threads 24. Similarly, lock mandrel 29 includes upper mandrel knob threads 31 which engage the internal threads 36 of locking knob 35. FIG. 5A also shows a guide cap 40 threading onto the upper mandrel knob threads 31 such that guide cap 40 can be partially recessed in a top cavity 45 of locking knob 35. Guide cap 40 further includes the funnel-shaped guide aperture 42 to assist in the initial insertion of the guidewire 75 into the top or proximal end of the surgical instrument. This guide aperture is “funnel-shaped” in the sense that it includes an aperture having an opening of greater diameter than that of the central passage, but then tapering to a diameter substantially the same diameter as that of the central passage. A lower funnel-shaped guide aperture 42 is seen on the lower end of lock mandrel 29 to assist in directing the guidewire 75 when it is traveling up the instrument central passage after being inserted into the bottom or distal end of the surgical instrument.

A further element of cannula lock 25 includes cam surface 37 formed on an internal portion of locking knob 35 together with ball apertures 32 formed in lock mandrel 29 between the lower mandrel threads 30 and the upper mandrel knob threads 31. Ball apertures 32 extend through the lock mandrel wall to communicate with mandrel central passage 34. Locking balls 27 are positioned in ball apertures 32, with the ball apertures 32 being sized such that a portion of the balls may extend into mandrel central passage 34 when the balls are fully seated in ball apertures 32. In the illustrated embodiment, there are three or four ball/ball aperture combinations spaced around the circumference of lock mandrel 29. However, fewer or more ball/ball apertures could be formed on lock mandrel 29, including just a single ball/ball aperture.

It may be envisioned from FIG. 5A how cam surface 37 on locking knob 35 will engage locking balls 27. When locking knob 35 is rotated, thereby moving toward or away from grip portion 21 of handle 20 on the upper mandrel knob threads 31, the cam surface 37 forces balls 27 to move more toward mandrel central passage 34 or allows balls 27 to move further away from mandrel central passage 34, respectively. Thus, rotating locking knob 35 to move cam surfaces 37 downward (toward grip portion 21) will force balls 27 to impinge on a guidewire in mandrel central passage 34, thereby “locking” the guidewire in place against longitudinal or axial movement along the central passage of the surgical instrument (however, “locking” does not necessarily need to preclude rotation of the guidewire within the central passage). Likewise, rotating knob 35 to move the cam surfaces 37 upward (away from grip portion 21) will allow balls 27 to move away from mandrel central passage 34 and “unlock” the guidewire to allow it to move freely in the central passage of the surgical instrument.

Although the particular cannula lock shown in FIG. 5A is one or more balls moving on a cam surface, the locking mechanism could include any type of impingement member which is capable of gripping the guidewire and holding it against relative movement along the length of the surgical instrument's central passage. For example, FIG. 5B shows the cannula lock 25 formed of a threaded channel 50 through which guide wire 75 passes. In this embodiment, the threaded channel 30 is formed as part of an end cap on the top of the instrument handle and the threaded channel 30 is oriented in a direction perpendicular to the main axis of the instrument shaft. The thumbscrew 51 engages threaded channel 50 and is advanced into guide wire 75 in order to move the cannula lock into its locked position. Alternatively, FIG. 5C suggests a cannula lock 25 formed by a locking knob 52 having external threads 53 and a series of collet fingers 54 extending below external threads 53. As best seen in FIG. 5D, the locking knob 52 will engage internal threads 55 form in the proximal end of shaft 4. When the locking knob 52 is advanced toward the distal end of shaft 4, the collet fingers 54 will ultimately encounter the cam surface 56 which forces collet fingers into engagement with guidewire 75. The FIGS. 5C and 5D illustrate an embodiment of cannula lock 25 which is formed on or continuous with shaft 4 (i.e., as opposed to being on the instrument handle).

FIGS. 5E to 5G suggests a cannula lock 25 formed by a cam lever 60 positioned in a notch 63 formed in the handle cap 59. It can be envisioned how cam lever 60 rotates on the pin 62 extending through handle cap 59. Cam lever 60 includes the cam surface 61 which selectively engages guidewire 75 when the cam surface is rotated to the locked position (FIG. 5G) and released guidewire 75 when the cam surface is rotated to the unlocked position (FIG. 5F). It will be apparent that this creates a cannula lock which does not require rotation of handle cap 59 to move the cannula lock between the locked and unlocked position. FIGS. 5H and 5I illustrate a slightly modified version of the previous embodiment where cam lever 65 is positioned in notch 67 and oriented to rotate in a plane perpendicular to the main axis of the instrument shaft. FIG. 5H shows cam lever 65 in the unlocked position and FIG. 5I shows cam lever 65 in the locked position. Another example of an impingement member not illustrated could be a constricting sleeve. The above examples of the cannula lock are merely illustrative and the cannula lock could take the form of many other conventional or future developed locking mechanisms.

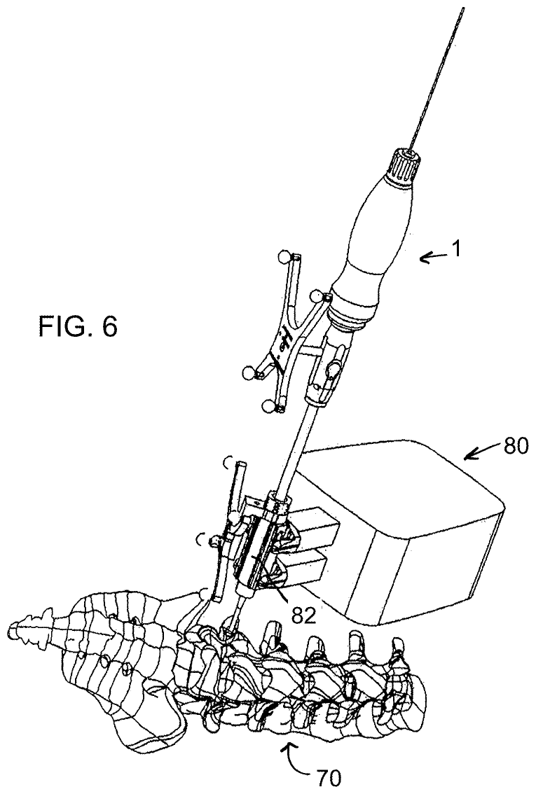

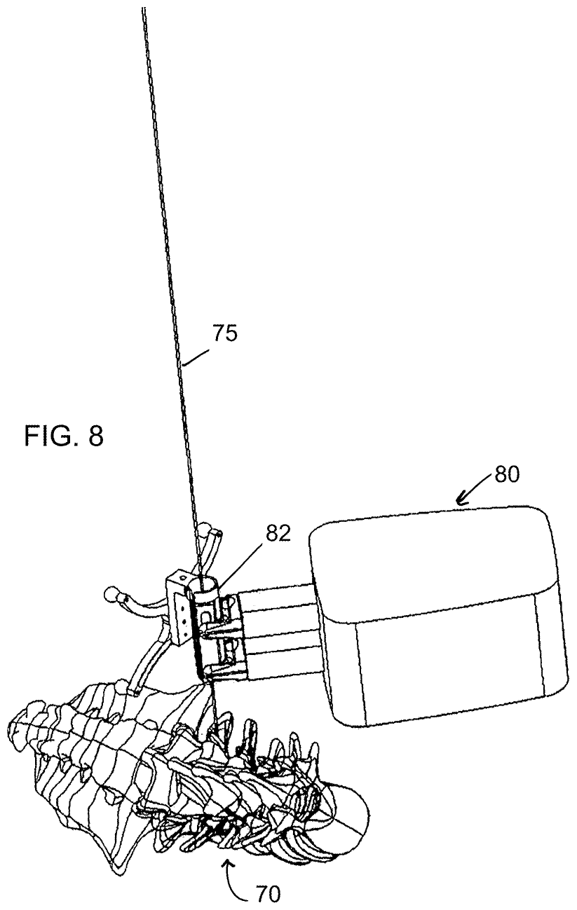

The distal end of surgical instrument 1 can take on different configurations to accomplish different surgical tasks, i.e., to function as different types of surgical instrument. For example, the distal end of surgical instrument 1 could be a relatively sharp point to form an awl. Alternatively, the distal end could have threads to form a tap (as shown in FIG. 7), or have a bit adapted to apply torque to a surgical screw in order to form a screw driver. Similarly, the surgical instrument could be a cannulated drill bit 73 such as shown in FIG. 9. In the FIG. 9 embodiment, the cannulated drill bit 73 has the type of cannula lock 25 shown in FIG. 5C.

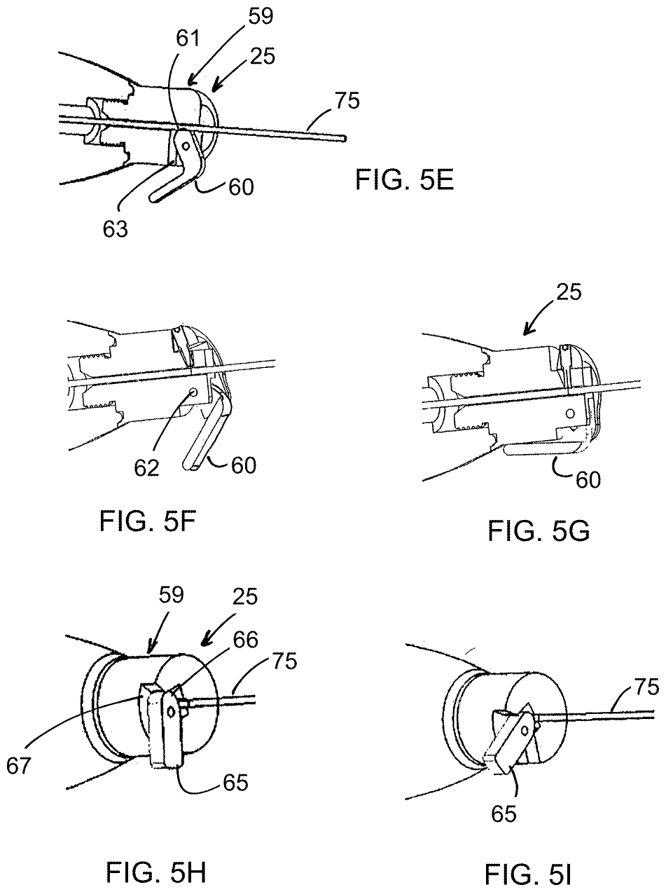

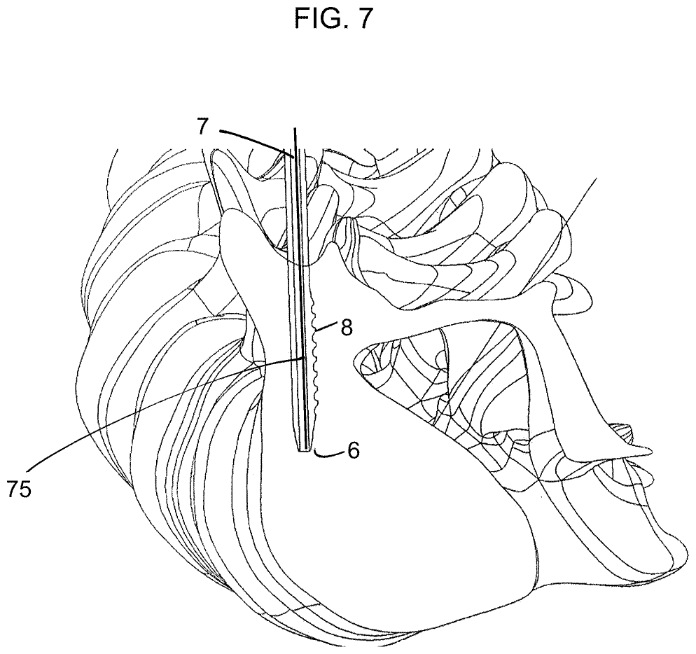

Those skilled in the art will recognize many different surgical procedures in which the above described surgical instrument may be employed. As suggested in the figures, the surgical instrument 1 could be used in conjunction with some type of surgical navigation system and in many cases include a robotic system 80, but it could also be used without a navigation system or a robotic system. In general, many procedures would include the initial step of, with the cannula lock in the unlocked position, inserting a guidewire into the central passage. The distal end of the guidewire can then be locked in any position the surgeon judges appropriate for the procedure at hand, but typically the guidewire will be locked in place with its distal end co-terminate with the distal end of the surgical instrument's cannula (e.g., see distal end of guidewire 75 shown in FIG. 7) or extending slightly beyond the distal end of the surgical instrument's cannula. In the method suggested by FIG. 6, the surgical instrument is inserted through the instrument guide 82 of the surgical robot 80 in order to direct the initial placement and alignment of the instrument relative to the intended trajectory involved with the surgical procedure step underway (e.g., advancing a tap into a previously formed bore in a vertebra). FIG. 7 suggests how the distal end of the instrument (in this case a tap with threads 8) is advanced into a bore previously formed in the vertebra by a tap and/or drill. Because the axial position of guidewire 75 in shaft cannula 7 is locked in place, there is no possibility of the guidewire extending further into the vertebra than its locked position. Once the tapping step is completed, guidewire 75 may be released by moving cannula lock 25 to an unlocked position in order to have guidewire 75 remain in the bore while the tap is backed out of the bore. The surgical instrument is then withdrawn by being slid over the guidewire which is being held stationary in the vertebra bore. At this stage, the guidewire appears as suggested in FIG. 8. Although not illustrated, a next step in the procedure could be to insert over (i.e., slide down) the guidewire a cannulated screw and a surgical instrument 1 which has a screw driver bit formed on its distal end. In this manner, the guidewire directs the screw into the bore in the vertebra for placement in the conventional manner. The screw typically will be advanced to its final positon (with the cannula lock 25 in its unlocked position) before the guidewire is removed.

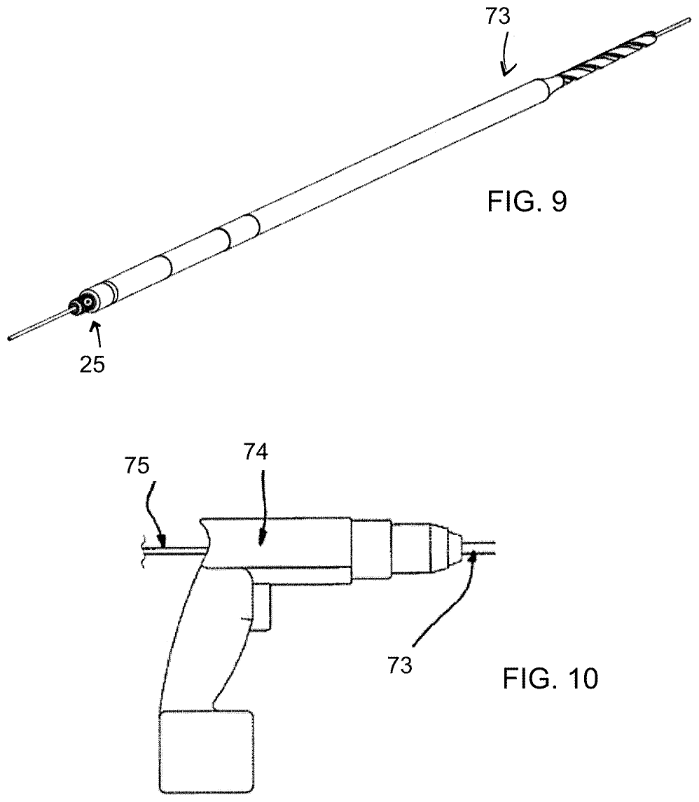

Many variations on the above described embodiments should be considered within the scope of the present invention. For example, while the drawings show cannula lock 25 being formed at the top of the handle (a member that drives the shaft manually), it could also be positioned along the middle of the handle or where the handle joins the shaft. Similarly, the cannula lock 25 could be positioned in or along the shaft in alternative embodiments. In another variation, the manually operated handle seen in the figures could be replaced with some type of powered or motorized tool or driving member. In one example, the powered (e.g., electric, pneumatic, or hydraulic) tool could be a cannulated drill 74 (see FIG. 10) used when the surgical instrument is a cannulated drill bit 73 (see FIG. 9). A similar cannulated driver could be used to apply torque to a tap shaft or awl shaft. The terms “cannulated driver” or “driving member” are used to describe any of a manually operated cannulated handle, a cannulated powered drill, or other powered, torque producing cannulated tool. The term “about” will typically mean a numerical value which is approximate and whose small variation would not significantly affect the practice of the disclosed embodiments. Where a numerical limitation is used, unless indicated otherwise by the context, “about” means the numerical value can vary by +/−5%, +/−10%, or in certain embodiments+/−15%, or even possibly as much as +/−20%. Similarly, “substantially” will typically mean at least 85% to 99% of the characteristic modified by the term. For example, “substantially all” will mean at least 85%, at least 90%, or at least 95%, etc.

1. A surgical bone tap comprising:
(a) a cannulated shaft having threads on a distal end;
(b) a cannulated handle attached to a proximal end of the cannulated shaft and forming a central passage extending through the cannulated handle and the cannulated shaft, the cannulated shaft being fixed to the cannulated handle such that there is no relative rotation there between; and
(c) a cannula lock configured to selectively move between (i) a locked position where a guidewire positioned in the central passage is engaged and cannot move axially with respect to the central passage, and (ii) an unlocked position where the guidewire is disengaged and can move axially with respect to the central passage.

2. The surgical bone tap of claim 1, wherein the cannula lock is continuous with the cannulated handle.

3. The surgical bone tap of claim 1, wherein the cannula lock is continuous with the cannulated shaft.

4. The surgical bone tap of claim 2, wherein the cannula lock is formed on a proximal end of the cannulated handle.

5. The surgical bone tap of claim 1, wherein the cannula lock includes an impingement member which moves at least partially into the central passage to engage the guidewire when in the locked position.

6. The surgical bone tap of claim 5, wherein the impingement member includes at least one of a ball, a cam, a thumbscrew, a pushbutton, a sleeve, or a collet.

7. The surgical bone tap of claim 6, wherein the cannula lock includes a cam surface moving the ball between the locked and unlocked position.

8. The surgical bone tap of claim 7, wherein the cannula lock includes (i) a lock mandrel connected to the handle and (ii) a locking knob having internal threads which travels on the lock mandrel.

9. The surgical bone tap of claim 7, wherein the cam surface is positioned on the locking knob.

10. The surgical bone tap of claim 7, wherein the lock mandrel is connected to the handle by way of external threads on the lock mandrel engaging internal threads in the handle.

11. The surgical bone tap of claim 1, wherein the handle includes a guide cap threaded onto a proximal end of the lock mandrel, the guide cap including a guide aperture having an opening of greater diameter than that of the central passage, but tapering to a diameter substantially the same as that of the central passage.

12. A cannulated surgical instrument comprising:
(a) a cannulated shaft;
(b) a cannulated driver attached to a proximal end of the cannulated shaft and forming a central passage extending through the cannulated driver and the cannulated shaft, the cannulated shaft being fixable to the cannulated driver such that there is no relative rotation there between; and
(c) a cannula lock configured to selectively move between (i) a locked position where a guidewire positioned in the central passage is engaged and cannot move axially with respect to the central passage, and (ii) an unlocked position where the guidewire is disengaged and can move axially with respect to the central passage.

13. The surgical instrument of claim 12, wherein the cannulated driver is either (i) a manually operated handle, or (ii) a powered cannulated drill.

14. The surgical instrument of claim 12, wherein the surgical instrument is one of (i) a tap, (ii) an awl, (iii) a screw driver, or (iv) a drill and drill bit.

15. The surgical instrument of claim 14, wherein the cannulated driver is a powered cannulated tool.

16-26. (canceled)

27. A method of selectively fixing a guidewire in a cannulated surgical instrument, the method comprising the steps of:
(a) providing the cannulated surgical instrument comprising:
(i) a cannulated shaft;
(ii) a cannulated handle attached to a proximal end of the cannulated shaft and forming a central passage extending through the cannulated handle and the cannulated shaft, the cannulated shaft being fixed to the cannulated handle such that there is no relative rotation there between; and
(iii) a cannula lock configured to selectively move between (1) a locked position where a guidewire positioned in the central passage is engaged and cannot move axially with respect to the central passage, and (2) an unlocked position where the guidewire is disengaged and can move axially with respect to the central passage;
(b) with the cannula lock in the unlocked position, inserting a guidewire through the central passage;
(c) moving the cannula lock to the lock position and performing a step in a surgical procedure;
(d) moving the cannula lock to the unlocked position and withdrawing the surgical instrument such that the central passage slides over the stationary guidewire.

28. The method of claim 27, wherein the surgical instrument is a bone tap and a distal end of the guidewire is first positioned in a bone aperture and then a proximal end of the guidewire is inserted into the central passage.

29. The method of claim 28, wherein the cannula lock is in the locked position while the bone tap is advanced into the bone aperture.

30. The method of claim 29, wherein after fully tapping the bone aperture, the cannula lock is placed in the unlocked position and the bone tap is withdrawn while leaving the guidewire inside the bone aperture.

31. The method of claim 30, wherein a cannulated bone screw is inserted over the guidewire and once the bone screw begins threaded engagement with the bone aperture, the guidewire is withdrawn from the bone aperture and bone screw.

32-41. (canceled)
